(19) United States
(12) Patent Application Publication
(10) Pub. No.: US 2026/0207795 A1
(43) Pub. Date: Jul. 23, 2026

(54) SMALL MOLECULE TRACERS BINDING TO ALPHA-SYNUCLEIN AGGREGATES AND THEIR USES

(71) Applicant: Fudan University, Shanghai (CN)

(72) Inventors: Yong CHU, Shanghai (CN); Jian WANG, Shanghai (CN); Jiang BIAN, Shanghai (CN); Xin LIN, Shanghai (CN); Yiqi LIU, Shanghai (CN); Chenyang QIU, Shanghai (CN); Jie HE, Shanghai (CN); Deyong YE, Shanghai (CN)

(21) Appl. No.: 18/717,525

(22) PCT Filed: Dec. 8, 2022
(86) PCT No.: PCT/CN2022/137518

§ 371 Date: Jun. 7, 2024

(30) Foreign Application Priority Data
Dec. 9, 2021 (CN) 202111498028.9

(51) Int. Cl. A61K 51/04; C07B 59/00; C07D 401/06; C07D 417/14

(52) U.S. Cl. CPC A61K 51/0459; C07B 59/002; C07D 401/06; C07D 417/14

(57) ABSTRACT

The present invention discloses a compound represented by Formula I shown below, a pharmaceutically acceptable salt thereof, or a solvate thereof, wherein R¹ is preferably pyridyl; R² is preferably halogen, halogenated C₁₋₄ alkoxyl; R³ and R⁴ are preferably methyl; ring A is preferably phenyl ring or thiazole ring.

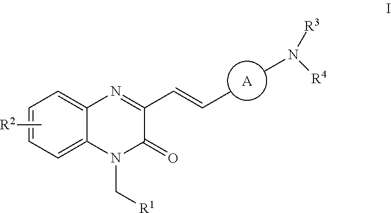

The compound of the invention can strongly and specifically bind to α-synuclein aggregates and so be used as a tracer for optical imaging of α-synuclein aggregates in biological samples or in vivo (such as brain). As radio-labeled the compound of the invention, it can be used as a radio imaging tracer for PET, SPECT, and other imaging techniques to realize the detection of α-synuclein lesions by non-invasive visualization in vivo (such as the brain). The compound of the invention is also used for preparing the radio-labeled tracer or its composition thereof. The α-synuclein diseases include Parkinson's disease, Parkinson's disease dementia, Alzheimer's disease, multiple system atrophy, dementia with Lewy body, etc.

### FIELD OF THE INVENTION

The present invention relates to novel compounds that are useful as tracers of α-synuclein aggregates and their use in the imaging of α-synuclein.

### BACKGROUND OF THE INVENTION

α-synuclein (α-Syn) lesions are an important pathogenesis of neurodegenerative diseases (Vekrellis, 2010). The abnormal aggregation of α-synuclein and the formation of Lewy bodies and Lewy neurites with its main components are the important pathological features and pathogenic factors of various neurodegenerative diseases including Parkinson's disease (PD), Parkinson's disease dementia (PDD), dementia with Lewy body (DLB), and multiple system atrophy (MSA). The process from the formation of α-synuclein deposition to the appearance of clinical symptoms is relatively long, usually lasting several years or even more than ten years, and it is too late to intervene when the patient has developed clinical symptoms. Early clinical intervention is very important to delay the progression of the disease and improve the quality of life and prognosis of patients. Therefore, the development of reliable early detection methods is the key point for the early diagnosis, prevention, and treatment of neurodegenerative diseases. At the same time, regulating the aggregation process of α-synuclein is also an important strategy for the treatment of these neurological diseases.

Due to its important role in the pathogenesis and progression of various neurodegenerative diseases, α-synuclein has become an important biomarker for early diagnosis of these diseases and an important target for drug therapy. However, the detection of α-synuclein aggregates currently can only be based on histological analysis of autopsy materials, and fail to make non-invasive detection in vivo. Molecular imaging is the best way to solve this problem.

Molecular imaging is based on the specific binding of molecular tracer probes (e.g., radioactive tracer probes, fluorescent tracer probes, etc.) to biomarkers (e.g., receptors, enzymes, ion channels, misfolded proteins), which are visualized by PET, SPECT, NMR, near-infrared, or other methods to provide diagnostic information in vivo. To realize molecular imaging, the key is to get a small molecule compound that can bind to a given molecular target as the imaging tracer probe. Since pathological changes of α-synuclein, Aβ, and Tau proteins are often co-deposited in neurodegenerative human brains, imaging probes of the specific protein must have not only a strong affinity for the target protein aggregate but also a high selectivity for abnormal accumulations of other proteins to achieve selective imaging. To date, few small molecule tracer probes have been reported that can visualize α-synuclein deposition in the brain of patients.

### DISCLOSURE OF INVENTION

The object of the present invention is to provide novel small molecule tracer probes capable of imaging α-synuclein aggregates, and radionuclide labeled small molecule tracer probes for imaging diagnosis of diseases related to α-synuclein accumulation, and processes for the preparation of said compounds, which can be useful to patients with neurodegenerative diseases such as Parkinson's disease, Lewy body dementia and multiple system atrophy for their in vivo non-invasive early diagnosis, disease monitoring and drug efficacy evaluation.

For the above purposes, the present invention provides compounds represented by Formula I, salts thereof, and solvates thereof. The compound has a strong affinity for α-synuclein aggregates, good selectivity for Aβ and Tau proteins, and good blood-brain barrier permeability. In particular, it can bind and stain Lewy bodies and Lewy neurites well specifically in patient brain tissue, and can be used as a fluorescence imaging tracer. Or it can be used as a radiographic imaging tracer required by PET, SPECT, and other imaging techniques to image α-synuclein pathology in vivo (such as the brain).

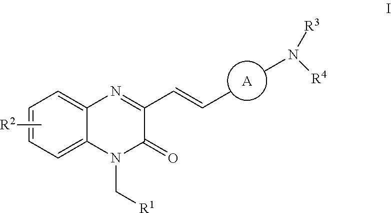

wherein,
R¹ is 5~6 membered aryl, preferably pyridyl;
R² is selected from halogen, nitro, hydroxyl, C₁₋₄ alkoxyl, and halogenated C₁₋₄ alkoxyl, wherein the halogen is selected from fluorine, chlorine, bromine, or iodine;
R³ and R⁴ are independently selected from hydrogen, and C₁₋₃ alkyl, preferably from methyl;
Ring A is selected from phenyl ring, 5- or 6-membered aromatic heterocyclic ring, preferably from phenyl or thiazole.

Wherein, one or more atoms of a compound of Formula I are the radioisotopes of that atom, preferably taken from ¹¹C, ¹³N, ¹⁵O, ¹⁸F, ⁷⁶Br, ¹²³I, ¹²⁵I, and ¹³¹I.

The invention also provides preparation methods for compounds of Formula I, which comprises the following synthesis routes:

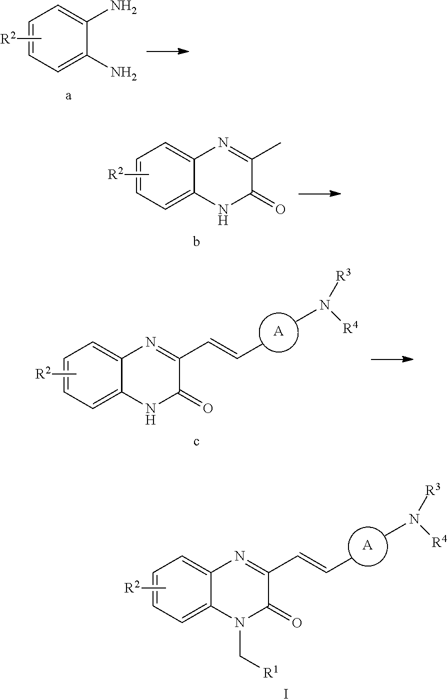

The compound of formula b is prepared by a ring-closed reaction of the compound of formula a at room temperature. Solvents used in the reaction include but are not limited to methanol, ethanol, dichloromethane, chloroform, triethylamine, dimethylformamide, tetrahydrofuran, and dioxane. Under acidic conditions and with concentrated sulfuric acid as a catalyst, the mixture of compound b and aldehyde with ring A is heated to form compound c. The solvents used are methanol, ethanol, triethylamine, dimethylformamide, tetrahydrofuran, dioxane, acetic acid, dichloromethane, and chloroform. The acids used are selected from organic acids (including but not limited to oxalic acid, malic acid, tartaric acid, citric acid, and acetic acid) and inorganic acids (hydrochloric acid, sulfuric acid, nitric acid, and trifluoroacetic acid); The reaction temperature ranges from 20° C. to 150° C., and the optimal reaction temperature is 80° C.-130° C. The compound of formula c undergoes the nitrogen substitution reaction under the action of base to obtain the compound of Formula I. The bases used include organic bases and inorganic bases. The organic bases include but are not limited to sodium bis(trimethylsilyl)amide, triethylamine, N, N-diisopropylethylamine n-butyl lithium, potassium t-butoxide, tetrabutylammonium bromide, and the inorganic bases include but are not limited to sodium hydride, sodium carbonate, sodium bicarbonate, potassium carbonate, potassium bicarbonate or cesium carbonate. The solvents used are selected from dichloromethane, tetrahydrofuran, dimethyl sulfoxide, dioxane, and dimethyl formamide. The reaction can happen in the temperature range of 20-120° C., and the optimal reaction temperature is 20° C.-40° C.

Further, the Formula I compound with R² as methoxy group is demethylated to form the Formula I compound with R² as hydroxyl group, and the hydroxyl group is oxygenated to form the Formula I compound with R² as halogenated alkoxy group.

The present invention also provides precursor compounds for the preparation of a labeled Formula I compound, whose structures are listed as follows:

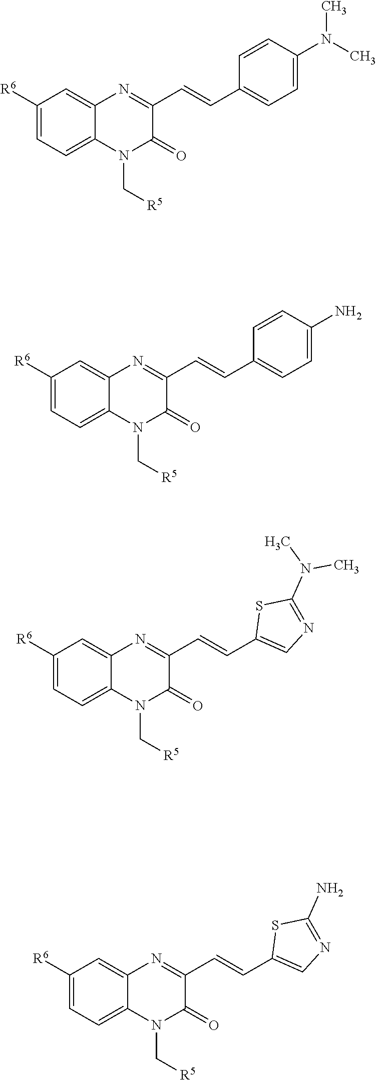

Wherein, R⁵ is pyridyl; R⁶ is independently selected from hydroxyl, fluorine, bromine, iodine, nitro, borate group, TsO—(CH₂)m-O—, MsO—(CH₂)m-O—, where m is an integer from 2 to 4.

One or more atoms in the Formula I compound can be labeled as a radionuclide by the precursor compounds mentioned above. Accordingly, the present invention also provides labeled Formula I compounds, preferably selected from the following structures:

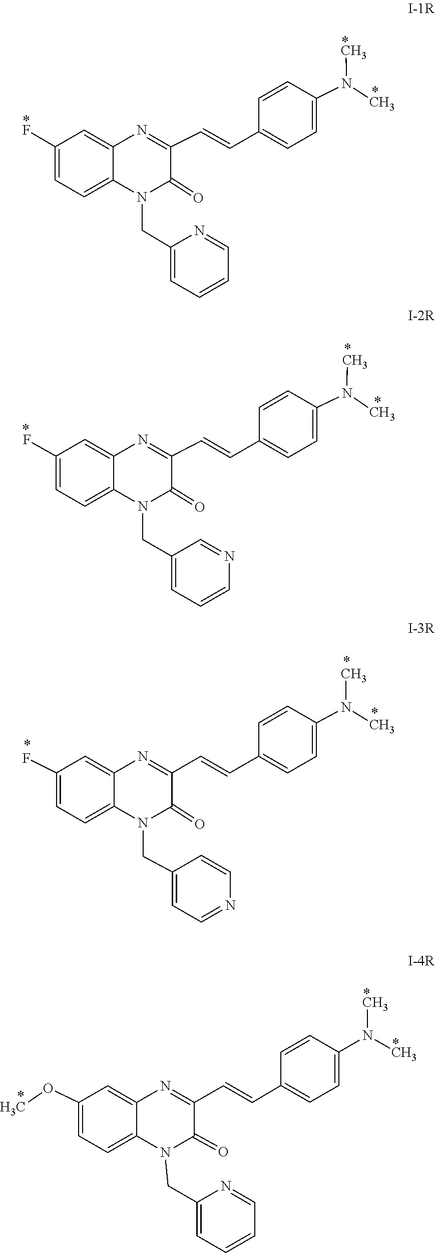

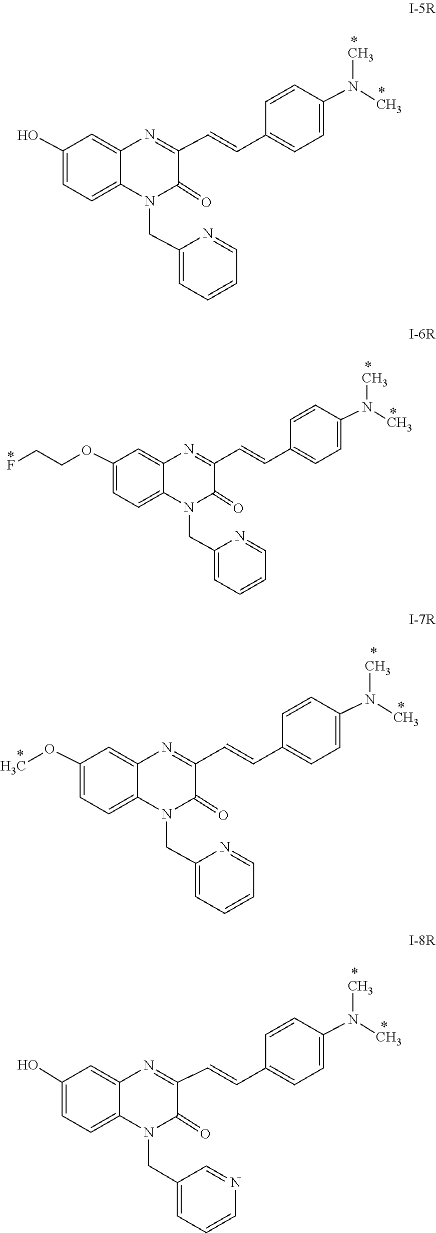

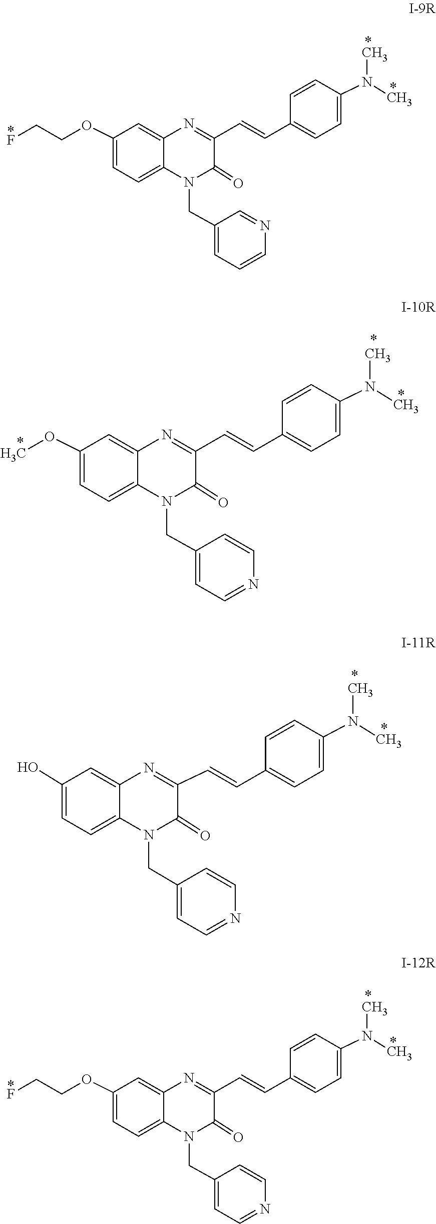

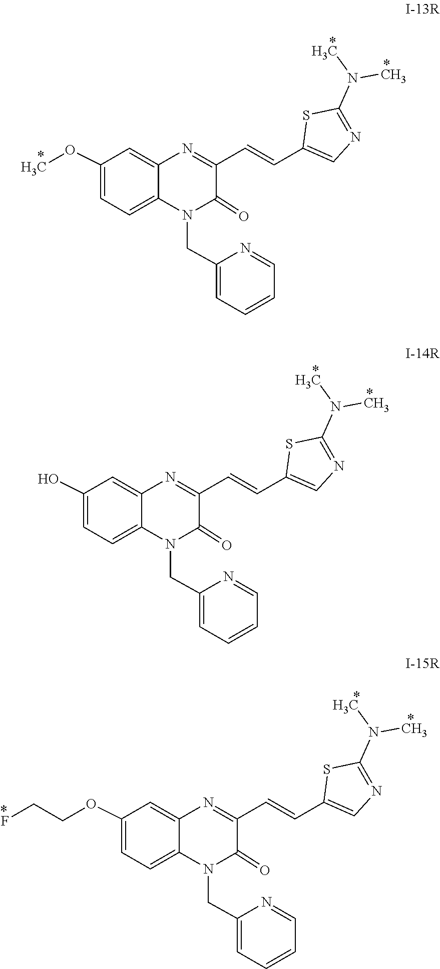

Wherein one of the atoms marked with * is a radioisotope of that atom at least.

The invention also provides the use of Formula I compounds that can specifically bind to α-synuclein aggregates. The compound can be used as a tracer for fluorescence imaging based on its self-fluorescence. As one or more atoms of the compound are replaced by radioactive atoms, they can be used as radiometric tracers required by various imaging techniques. For example, when fluorine or carbon atoms of the compound are replaced by radionuclides ¹⁸F or ¹¹C, they can be used as radiometric tracers for imaging of Positron Emission Tomography (PET), or for the preparation of such imaging tracers or composition with it. These imaging tracers can be used to detect neurological diseases associated with α-synuclein misfolding and aggregation, to screen for therapeutic or preventive drugs for diseases associated with α-synuclein aggregates in the brain, or to quantify or determine the accumulation of α-synuclein aggregates in the brain.

### THE BENEFICIAL EFFECTS OF THE INVENTION

Positron Emission Tomography (PET) and Single-Photon Emission Computed Tomography (SPECT) are the most advanced non-invasive 3D imaging techniques. The use of PET and SPECT radiotracer that binds specifically to a given biologic target can provide in vivo real-time diagnostic information closest to pathology to prove and quantify pathophysiological changes related to the disease, and is the most powerful tools for early clinical diagnosis, monitoring disease progression, and developing therapeutic drug. Radionuclides used for PET generally include ¹¹C, ¹³N, ¹⁵O, and ¹⁸F, with radioactive half-lives of 20, 10, 2, and 110 minutes, respectively. ¹⁸F is usually the best choice as a radionuclide for PET because it has the longest half-life and so is the most convenient to use. In addition, ^{99m}Tc, ¹²³I, ¹³¹I, and ¹¹¹In are the radionuclides most commonly used in SPECT. In principle, these nuclides can be used to replace any corresponding non-radioactive isotope atom in the probes to make it radioactive.

Therefore, when a radionuclide is labeled to a specific probe of α-synuclein aggregates, it can be used as a tracer for autoradiography in vitro or PET/SPECT imaging in vivo to achieve visualization of pathological α-synuclein. It has greatly facilitated the diagnosis, management, mechanism research, and development of therapeutic drugs for neurological disorders associated with α-synuclein misfolding and aggregation. The key to realizing imaging is to find probes with high affinity and selectivity to α-synuclein and to label them with radionuclides as imaging tracers for PET and SPECT. The present invention provides a novel type of compounds that have strong affinity and high specificity for binding of α-synuclein aggregates and can cross the blood-brain barrier. These compounds exhibit autofluorescence and make a clear fluorescent staining indicating that they can specifically bind to the α-synuclein fibers in cell models, as well as Lewy bodies and Lewy neurites in patient brain tissue (the main component of which is α-synuclein aggregates). Thus the compounds can be used as fluorescent imaging tracers for α-synuclein detection. As one or more fluorine or carbon atoms of said compounds in the present invention are replaced with radionuclides ¹⁸F or ¹¹C, they can be used as radio-imaging tracers of autoradiography or PET for imaging α-synuclein aggregates in vitro or in vivo, especially in the brain. When halogen atoms in the compound of the invention are replaced with radioactive isotopes of iodine or other acceptable nuclides, they can be used as tracers for SPECT to image α-synuclein aggregates.

The invention also provides processes for the preparation of Formula I compounds and radio-labeled compounds thereof, as well as precursor compounds for the preparation of radio-labeled compounds and preparation methods thereof. Further, the invention also provides methods of diagnostic imaging and quantifying or determining α-synuclein accumulation in the brain, as well as drug screening for preventing or treating α-synuclein accumulation diseases by Formula I compounds or their compositions thereof.

### A BRIEF DESCRIPTION OF THE FIGURES

FIG. 1 is the con-focal laser microscope photograph of immunofluorescence staining of α-synuclein aggregates in a SH-SY5Y cell model by a compound of the invention. The white triangle represents the signal of the compound co-located with the α-synuclein antibody, the white arrow represents the non-specific staining signal of the compound, the red arrow represents the signal of α-synuclein antibody that the compound failed to bind, and the yellow arrow represents the signal of the compound that failed to dissolve.

FIG. 2 is a fluorescence microscope photograph of the compound of the invention to stain the brain slice of a patient with Lewy body dementia (DLB). White arrows indicate Lewy bodies (left) or Lewy neurites (right). The result shows that the compound can strongly bind to the α-synuclein aggregates in the patient's brain.

FIG. 3 is a fluorescence microscope photograph of the compound of the invention to stain the brain slice of a patient with Alzheimer's disease (AD). The white arrow represents the original Aβ plaque, the white triangle represents the Aβ dense core plaque, and the yellow triangle represents the Tau neurofibrillary tangles. The result shows that the compound has good target selectivity in patient tissue with very weak binding to the Aβ and Tau lesions.

### THE BEST WAY TO REALIZE THE INVENTION

In this description, “α-synuclein accumulation disease” refers to diseases in which α-synuclein is abnormally folded and accumulated in the brain, including but not limited to Parkinson's disease (PD), Parkinson's disease dementia (PDD), multiple system atrophy (MSA), Lewy body dementia (DLB), etc. The present invention provides the Formula I compound, its salt or solvate thereof as an imaging tracer to visualize α-synuclein in vivo or in vitro to give diagnostic and evaluation information for α-synuclein accumulation diseases.

In the present invention, the compounds that can be used for imaging α-synuclein accumulation are represented by general Formula I, or its salts and solvates thereof. The compound of the invention has a double bond between two rings so the Formula I compound can be cis-isomers, trans-isomers, and both of them. The preferred compounds are I-1, I-2, I-3, I-6, I-12, I-15. In particular, I-15 can well label the α-synuclein in Lewy bodies and Lewy neurites from the DLB patient's brain tissue, and show good specificity with very weak binding to the Aβ and Tau lesions in the brain tissue of patients with Alzheimer's disease (AD).

The invention also includes salts of Formula I compounds which nitrogen atoms can be used to form the pharmaceutically acceptable salts.

Any chemical formula given in the present invention is also intended to represent a form of the compound with isotopic labeling. The isotopically labeled compounds have the structure shown in Formula I, differing only in that one or more of the atoms are replaced by its radioisotope. Isotopes that may be incorporated into the compounds of the invention include isotopes of hydrogen, carbon, nitrogen, oxygen, fluorine, chlorine, and iodine, such as ²H, ³H, ¹¹C, ¹³C, ¹⁴C, ¹⁵N, ¹⁸O, ¹⁷O, ³⁵S, ¹⁸F, ³⁶Cl, ¹²³I, ¹²⁵I, and ¹³¹I, respectively. Substituting with heavier isotopes (such as deuterium, ²H) can provide certain advantages arising from greater metabolic stability (such as increased in vivo half-life or reduced dose requirements). Substitution with ²H can be used specifically to prevent the formation of unwanted radio metabolites or to block radiodefluorination. When used to label the compound of the invention, positron radionuclides like ¹¹C, ¹³N, ¹⁵O, and ¹⁸F are preferred for PET imaging, in which ¹⁸F is the most preferred and ¹¹C next. Additionally, ¹²³I is preferred among y radionuclides for SPECT imaging.

The invention also includes radio-labeled Formula I compounds. In theory, any sites at the compound of Formula I can be labeled by a radionuclide, but it is preferable to replace the halogen, nitro, or label the alkyl group as shown in the examples. For example, when the compounds of the present invention are labeled with ¹⁸F, any location of the compound can be labeled, preferably replacing nitro or fluorine atoms.

The radiolabeled compounds of the present invention and their precursors required for a label can generally be prepared by conventional processes and schemes disclosed in examples, or by the following preparation methods (substituting non-isotopic labeling reagents by readily available isotopic labeling reagents). Some methods have been reported to label ¹¹C, ¹⁵N, ¹⁸O, ¹⁸F, or other isotopes to compounds (Angew. Chem. Int. Ed. Miller, Philip W, 2008, 47, 8998-9033; Peter J. H. Scott, 2009, 48, 6001-6004; Chem. Rev., Sean Preshlock, 2016, 116, 719-766; Frederic Dollé, Fluorine-18 chemistry for molecular imaging with positron emission tomography. Fluorine and Health: Molecular Imaging, Biomedical Materials and Pharmaceuticals (Tressaud, A. Haufe, G.), 2008, pp. 3-66, Elsevier). The Formula I compounds with radionuclide-labeled can be used as PET or SPECT tracers for imaging α-synuclein accumulations in vivo.

The present invention also provides precursor compounds for preparing radionuclide-labeled Formula I compounds. A person skilled in the art may design and synthesize the precursor compound according to the structure shown in the invention. That is, the precursor compound can be obtained by structurally modifying the commercially available compound or the compound of the present invention.

The radio-labeled compounds of the invention can be prepared by different precursor compounds. Typically, the labeled sites of precursors from Formula I compounds contain hydroxyl or nitro, bromine, iodine, borate, or other leaving groups (such as MsO—, TsO—, etc.), which can be substituted with ¹¹C or ¹⁸F, respectively. In particular, the precursor compound containing the hydroxyl can be obtained by removing the methyl from the methoxyl, and then directly labeled with ¹¹C, or produce ¹⁸F—CH₂CH₂—O-substituted compounds to achieve radioactive labeling by oxo-alkylation reaction of ¹⁸F-labeled brominated alkanes like ¹⁸F—CH₂CH₂—Br. Similarly, the precursor compound may also contain bromine, iodine, borate, and leaving groups of TsO— or MsO—, which can be replaced by ¹⁸F by well-known methods. For example, precursor compounds that can be used to prepare radiotracers include I-5 (the precursor of I-4 and I-6), I-8 (the precursor of I-7 and I-9), I-11 (the precursor of I-10 and I-12), I-14 (the precursor of I-13 and I-15), etc. In the synthesis of compounds such as I-6, I-9, I-12, I-15, the position to be labeled in the precursor compound is preferentially converted to be leaving groups of TsO— or MsO—.

Generally, the nuclides used for labeling are produced by cyclotrons, and a skilled person in the field may choose the appropriate method and instrument according to the nuclide to be manufactured. The methods of labeling are known in the field and mainly include chemical synthesis, isotope exchange, and biosynthesis. The radio-labeled compound of the invention can be administered locally or systematically to the patient, and after sufficient time of binding and dissociation with α-synuclein, the detection site can be visualized by PET and SPECT. The administration can be subcutaneous, abdominal, intravenous, arterial or spinal fluid injection or infusion, or oral, with full attention to the patient's exposure dose, depending on factors like the type of disease, the nuclide labeled, the compound used, the patient's condition, the difference of test site, etc.

The present invention also provides compositions for imaging diagnosis of α-synuclein accumulation diseases, which comprise compounds of the present invention, pharmaceutically acceptable salts thereof, or solvates thereof, and pharmaceutically acceptable carriers. The preferred composition comprises the labeled compounds of the present invention, where labeling with radionuclides (in particular positron radionuclides ¹¹C, ¹³N, ¹⁵O, ¹⁸F, etc.) is preferred for in vivo imaging diagnostics. Depending on its use, the compound or its composition thereof is preferably a form of injection for application. Therefore, pharmaceutically acceptable carriers are preferred to be liquid, including (but not limited to) aqueous solvents (such as potassium phosphate buffers, salt water, Ringer's solution, and distilled water) or anhydrous solvents (such as polyethylene glycol, vegetable oil, ethanol, glycerin, dimethyl sulfoxide, and propylene glycol). The proportion of the carrier and the compound of the invention can be appropriately varied, depending on the site of action, detection means, etc. In addition, the composition thereof may include commonly used antimicrobials (such as antibiotics, etc.), local anesthetics (such as procaine hydrochloride, ibucaine hydrochloride, etc.), buffers (such as trihydrochloric acid buffers, HEPES buffers, etc.), osmotic pressure regulators (such as glucose, sorbitol, sodium chloride, etc.).

The compounds of the invention include labeled or unlabeled ones which can be labeled before use by the methods described above.

The compound of the invention can bind to α-synuclein highly and specifically and therefore be used for staining and quantification of α-synuclein in vitro through labeled or unlabeled compounds. For example, due to their self-fluorescence, the compounds can be directly used to stain α-synuclein in a specimen and observe the fluorescence results by laser confocal or fluorescence microscopy or colorimetric to quantify α-synuclein. A scintillation counter is used for the quantification of α-synuclein after radiolabeling of the compounds. The early pathological of synuclein diseases such as Parkinson's disease (PD), dementia with Lewy body (DLB), multiple system atrophy (MSA), etc., is the formation of Lewy bodies, in which the main component is the abnormal accumulation of α-synuclein, and so the detection of α-synuclein can provide the early onset information of these diseases. Because the compound of the invention can stain Lewy bodies and Lewy neurites, it can be used to study the pathological mechanism and the diagnosis of patients in the clinic. Staining brain sections using compounds of the invention can be performed by common methods. As mentioned above, the compounds of the present invention, i.e. the compounds shown in Formula I and their salts or solvates thereof, can be used as imaging tracers for α-synuclein accumulations, preferably radionuclide labeled imaging tracers.

Therefore, the present invention provides:
Formula I compounds, and their pharmaceutically acceptable salts or solvates thereof, used as tracers for imaging α-synuclein accumulations;
Optical and radioactive tracers for imaging diagnosis of α-synuclein accumulation diseases, in particular, positron radionuclide labeled imaging tracers;
The precursor compounds for the preparation of radioisotopically labeled Formula I compounds;
A composition for the imaging diagnosis of α-synuclein accumulation diseases comprising a compound of Formula I, or its pharmaceutically acceptable salt or solvate thereof, and pharmaceutically acceptable carrier;
The use of compounds of Formula I, their pharmaceutically acceptable salts, and solvates thereof for the imaging diagnosis of α-synuclein accumulation diseases;
The use of compounds of Formula I, their pharmaceutically acceptable salts, and solvates thereof in the production of compositions for the imaging diagnosis of α-synuclein accumulation diseases.

In addition, the present invention provides:
Methods for detection/staining of α-synuclein aggregates in brain samples, and Lewy bodies and Lewy neurites in patients' brains, which can be used to provide information for early diagnosis and assessment of progression of diseases with α-synuclein accumulation;
Methods for quantifying or determining the accumulation of α-synuclein in the brain; Screening methods for drugs for the prevention and/or treatment of α-synuclein accumulation diseases;
In all the above methods, it is used of Formula I compounds, or their pharmaceutically acceptable salts or solvates thereof with pharmaceutically acceptable carriers.

In the following, substituents of Formula I compounds are explained, and salts, solvates, and derivatives of Formula I compounds are explained, as well as the labeling methods.

### Definition

Unless otherwise indicated, the meaning and scope of the terms of the present invention are described and limited as defined below.

The terms “compound of Formula I”, “Formula I compounds”, or “compound of the present invention” refer to any compound selected from a class of compounds represented by Formula I, including its stereoisomers, cis-trans isomers, tautomers, solvates, and salts (e.g., medicinal salts).

Unless otherwise specified, the use of “or” or “and” means “and/or”.

When indicating the number of substituents, the term “one or more” means the number of substituents from one to the largest chemically possible number, i.e., substituting one hydrogen to all hydrogens by a substituent.

The term “substituent” refers to an atom or group of atoms that replaces the hydrogen atoms on the parent molecule.

The term “halogen” refers to fluorine (F), chlorine (Cl), bromine (Br), and iodine (I).

The term “TsO—” means structure as

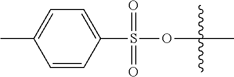 [SMILES: *OS(=O)(=O)c1ccc(C)cc1]

and “MsO—” means structure as

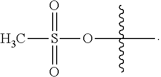 [SMILES: *OS(C)(=O)=O]

The term “C₁₋₄ alkoxyl” denotes the group of the formula —O—R′, where R′ refers to a univalent straight or branched saturated alkyl group containing 1 to 4 carbon atoms, for example, methoxy.

The term “halogenated C₁₋₄ alkoxyl” denotes an alkoxy group in which one or more hydrogen atoms have been replaced by the same or different halogen atoms (especially fluorine atoms), for example, 1-fluoroethoxy.

The term “C₁₋₃ alkyl” refers to univalent linear or branched-chain saturated hydrocarbon groups with 1 to 3 carbon atoms, for example, methyl.

The term “5~6-membered aromatic heterocyclic ring” refers to an aromatic mono-heterocyclic ring with 5 or 6 atoms, consisting of 1, 2, 3, or 4 heteroatoms selected from N, O, and S with the remaining ring atoms being carbon, for example, thiophene.

The term “aromatic” is denoted by the conventional concept of aromaticity defined in the literature, especially IUPAC-Directory of Chemical Terms, 2nd Edition, A. D. McNaught & A. Wilkinson. Blackwell Scientific Publications, Oxford (1997).

The term “pharmaceutically acceptable salt” refers to salt that is not harmful to mammals, especially humans. Pharmaceutically acceptable salts can be formed by using non-toxic acids or bases, including inorganic or organic acids or bases, which include metal salts formed from aluminum, calcium, lithium, magnesium, potassium, sodium and zinc; Or with lysine, N, N′-dibenzyl ethylenediamine, chloroprocaine, choline, diethanolamine, ethylenediamine, meglumine (that is, N-methylglucamine) and procaine and other organic salts. In addition, the salts include acid-addition salts and alkali-addition salts.

The term “pharmaceutically acceptable carrier” means a saline solution, a pharmaceutically acceptable material, composition, or excipient such as a liquid or solid filler, diluent, solvent, or encapsulation material. Pharmaceutically acceptable carriers include but are not limited to water, salt solution, saline or phosphate buffered salt solution (PBS), sodium chloride injection, Ringer's injection, glucose injection, sterile water injection, glucose, and lactate Ringer's injection.

The term “solvate” refers to a solvent-containing compound formed by the association of one or more solvent molecules with the compound. For example, it may contain one solvate, two solvates, three solvates, and four solvates. In addition, solvates include hydrates.

The term “hydrate” refers to a compound or its salts containing water bound by non-covalent intermolecular forces, the amount of water contained may be stoichiometric or non-stoichiometric. For example, it contains monohydrate, dihydrate, trihydrate, and tetrahydrate.

### [Tracer Probe of α-Synuclein Aggregates]

The present invention provides a tracer of α-synuclein aggregates (hereinafter also referred to as a tracer), i.e. a compound of Formula I showed below, a pharmaceutically acceptable salt thereof, or a solvate thereof.

In addition, the Formula I compounds shown below have spontaneous fluorescence and be directly used as a tracer for optical imaging of α-synuclein aggregates. After one or more atoms in the compound are labeled with the radioisotopes of that atom, it can be used for radio-imaging α-synuclein aggregates by PET and SPECT.

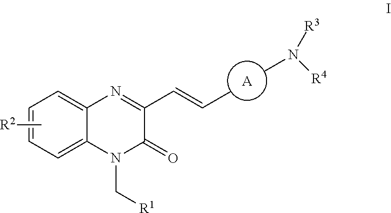

wherein,
R¹ is 5~6 membered aryl, preferably pyridyl;
R² is selected from halogen, nitro, hydroxyl, C₁₋₄ alkoxyl, and halogenated C₁₋₄ alkoxyl, wherein the halogen is selected from fluorine, chlorine, bromine, or iodine;
R³ and R⁴ are independently selected from hydrogen, and C₁₋₃ alkyl, preferably from methyl;
Ring A is selected from phenyl ring, 5- and 6-membered aromatic heterocyclic ring, preferably from phenyl or thiazole.

Wherein, one or more atoms of a compound of Formula I are the radioisotopes of that atom, preferably taken from ¹¹C, ¹³N, ¹⁵O, ¹⁸F, ⁷⁶Br, ¹²³I, ¹²⁵I, and ¹³¹I.

For example, the following compounds may be cited as examples of Formula I compounds shown below:

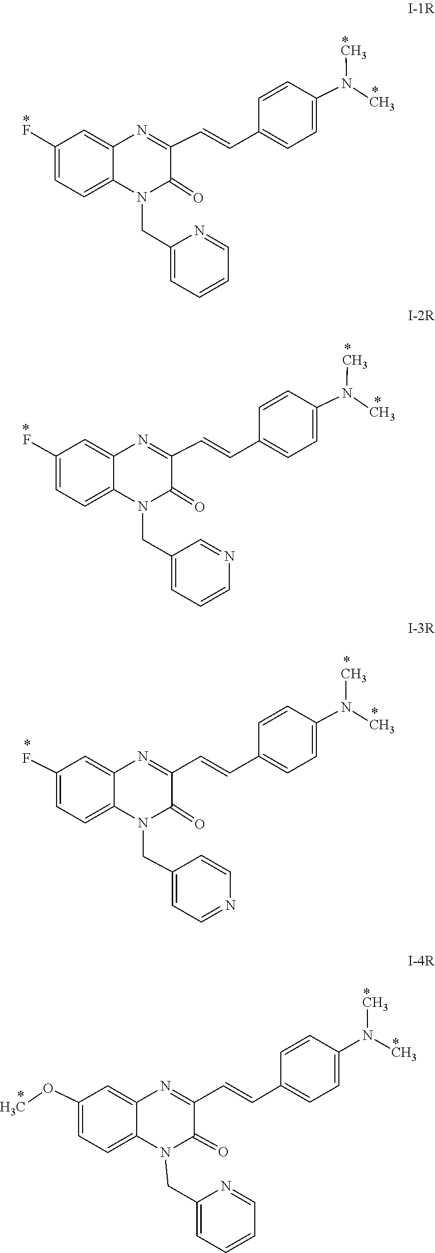

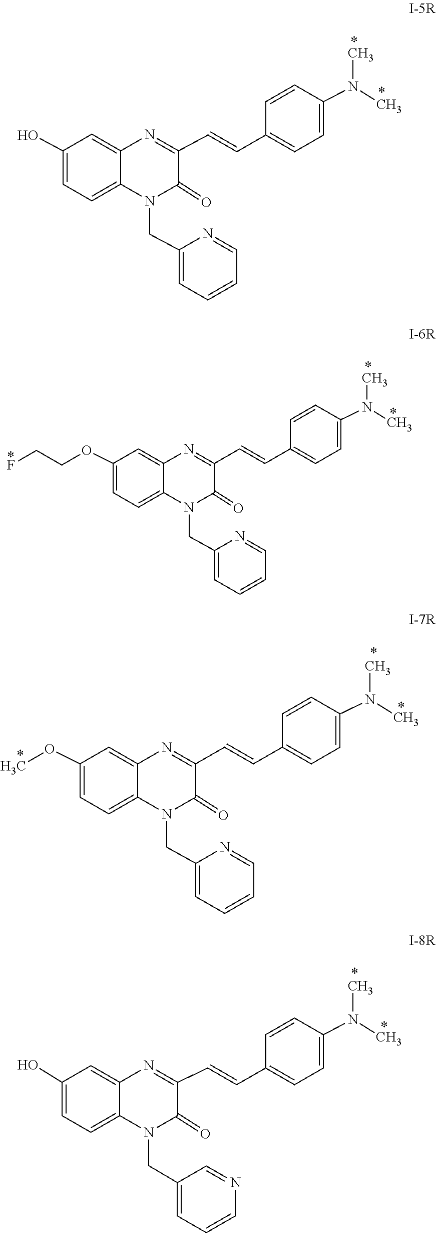

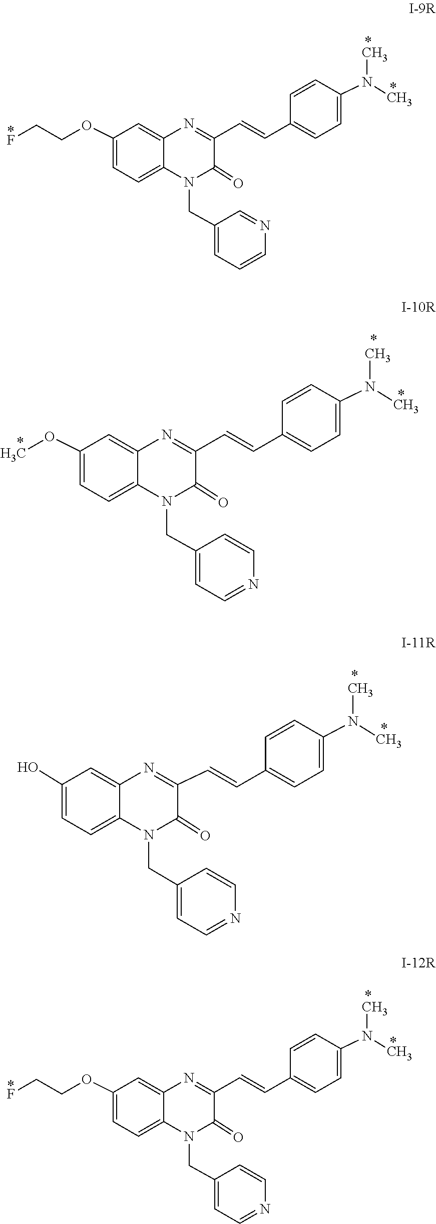

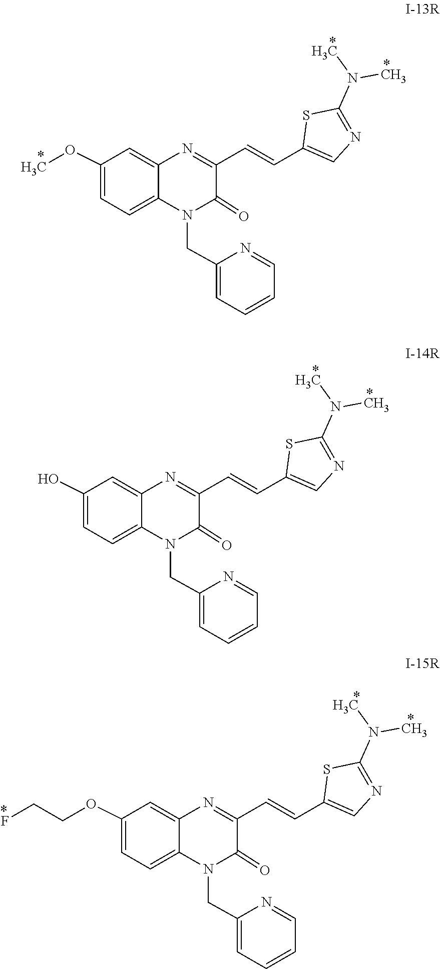

Any atom marked with * shown in the structures above may be a radioisotope of that atom, such as ¹¹C or ¹⁸F. Preferably, the *F in the compound is the radioisotope ¹⁸F, and the *C of the methoxy or dimethylamino group attached to the aryl group is the radioisotope ¹¹C.

In this specification, the meaning of the designation as (¹⁸F) I-15 is the structure named I-15 with the * marked atom ¹⁸F; In the same way, the meaning of the named (¹¹C) I-15 means that the atom marked with * in the structure numbered I-15 is ¹¹C.

### [Composition for Optical Imaging of α-Synuclein Aggregates]

Compositions of the present invention for optical imaging of α-synuclein aggregates (also hereinafter referred to as optical imaging compositions) comprise compounds of Formula I, pharmaceutically acceptable salts thereof, or solvates thereof. Optical imaging includes biological imaging in vitro and in vivo, which include but are not limited to the fluorescence microscopy method, multi-photon imaging method, two-photon imaging method, and near-infrared fluorescence imaging method.

### [Composition for Radiographic Imaging of α-Synuclein Aggregates]

A radiolabeled composition of the present invention for radiographic imaging α-synuclein aggregates (also hereinafter referred to as a radiographic imaging composition) comprises a radiolabeled Formula I compound, a pharmaceutically acceptable salt thereof, or a solvate thereof. Radiographic imaging includes imaging in vitro and in vivo, which include but are not limited to autoradiography, PET, and SPECT.

Compositions for optical imaging or radiographic imaging may be contained in the pharmaceutically acceptable carrier. The content of Formula I compounds contained therein, their pharmaceutically acceptable salts, or their solvates, and pharmaceutically acceptable carriers are not specified and may be adjusted according to many factors such as the compound used and age, weight, health status, sex, and content of the diet of the mammal being given; the frequency and means of giving; treatment period; and other agents used at the same time.

[Diagnostic Agents for Diseases Associated with α-Synuclein Aggregates, or Accompanying Diagnostic Agents for the Treatment or Prevention of Said Diseases]

A diagnostic drug of the invention for an α-synuclein aggregates-associated disease, or a concomitant diagnostic drug for the treatment or prevention of the disease (hereinafter also referred to as a concomitant diagnostic drug) comprises a compound of the invention. A therapeutic concomitant diagnostic drug is a diagnostic drug used to determine whether a treatment is likely to be implemented in the presence of the disease. In addition, prophylactic diagnostic drugs refer to diagnostic drugs used to predict the future onset of the disease or to determine whether it is possible to implement preventive disease suppression when the precursor symptoms of the disease are identified.

Based on the comparison of the imaging data obtained by the diagnostic agent between the amount and/or distribution of α-synuclein aggregates in the subject's body (e.g., brain) and the previously known correlations in the disease, it enables the subject to be diagnosed with the disease (specifically, such as whether they suffer from the diseases, severity, likelihood of onset, etc.), and better understand the disease status of the subject for formulating the prevention/treatment plan (type and combination of prophylactic administration/treatment drugs, dosage, usage, etc.).

### [Optical Imaging Method]

The optical imaging method of the invention comprises the following steps, which are illustrated in the following example of detecting α-synuclein aggregates in the brain, and similar methods for detecting other parts.

An effective amount of the tracer of the present invention is given to the tested organism, and the tracer that reaches the brain of the organism binds to the α-synuclein aggregates in the brain. Optical imaging (imaging) of the α-synuclein aggregates is then achieved by irradiating the first wavelength of light from outside the brain used to excite the tracer and detecting the second wavelength of light (e.g. fluorescence) emitted from the intracranial tracer. Wherein, the tracer contains a compound represented by Formula I, or a pharmaceutically acceptable salt thereof, or a solvate thereof.

### [Radiographic Imaging Method]

The radiographic imaging method of the invention comprises the following steps, which are illustrated in the following example of detecting α-synuclein aggregates in the brain, and similar methods for detecting other sites.

An effective amount of radiolabeled tracer of the present invention is given to the tested organism and will bind to the α-synuclein aggregates when reaching the brain. The radiation emitted from the tracer is then detected, enabling radiographic imaging (imaging) of the α-synuclein aggregates in the brain. Wherein the tracer contains a compound represented by Formula I, or a pharmaceutically acceptable salt thereof, or a solvate thereof, where one or more atoms of the compound of Formula I are radioisotopes of that atom.

The optical and radiological imaging subjects include mammals such as humans, rats, mice, rabbits, guinea pigs, hamsters, monkeys, dogs, minks, or miniature pigs. Preferably, mammals are humans. The tracer can be given orally, intravenously, or peritoneally without special limitation. Intravenous or intraperitoneal injection is preferred, and intravenous injection is most preferred.

### [Method for Quantifying or Determining the Accumulation of α-Synuclein in the Brain]

By calculating the difference in the amount and/or distribution of light or radiation detected in the subject organism (e.g., brain) to the normal mammals, the accumulation of α-synuclein in the body (e.g., brain) can be quantified and the presence or absence of α-synuclein aggregates in the body (e.g., brain) can be determined.

[Screening Methods for Therapeutic or Prophylactic Drugs to Prevent or Treat Diseases Associated with α-Synuclein Accumulation]

Based on the imaging method described above of [optical imaging method] and [Radiological imaging method], the light or radiation emitted by the tested organism is detected before and after administration of the screening drug, and the change of α-synuclein accumulation is determined according to the difference in its intensity and/or distribution for screening therapeutic or preventive drugs. For example, if the amount (intensity) of light (such as fluorescence) or radiation from the tracer is reduced after administration of the screening drug, the screening drug may be potentially used as a therapeutic or prophylactic drug for the disease or symptom. Preferably, if the amount and/or distribution of light or radiation from the animal model treated with the screening drug is close to normal animals (preferably mammals), the screening drug may have a better chance of therapeutic or preventing the disease or symptom.

The type of organism tested and the method of administration are the same as described above [Optical imaging method] and [Radiological imaging method].

The compounds of the present invention can be synthesized from known materials (e.g. commercially available materials) by a known method. A person skilled in the art may appropriately select starting materials and methods to synthesize the required compounds of the invention. The invention is further described below with examples, and it is understood that these examples are intended only to illustrate the invention but not to limit the scope of protection of the invention. Experimental methods not specified in the following examples are usually performed under general conditions or as recommended by the manufacturer. The known starting material of the invention may be prepared by a method known in the field, or purchased from a commercially available product. The structure of the compound is determined by nuclear magnetic resonance spectroscopy (NMR) and/or mass spectrometry (MS).

Example 1: Preparation of compound I-1, as shown below:

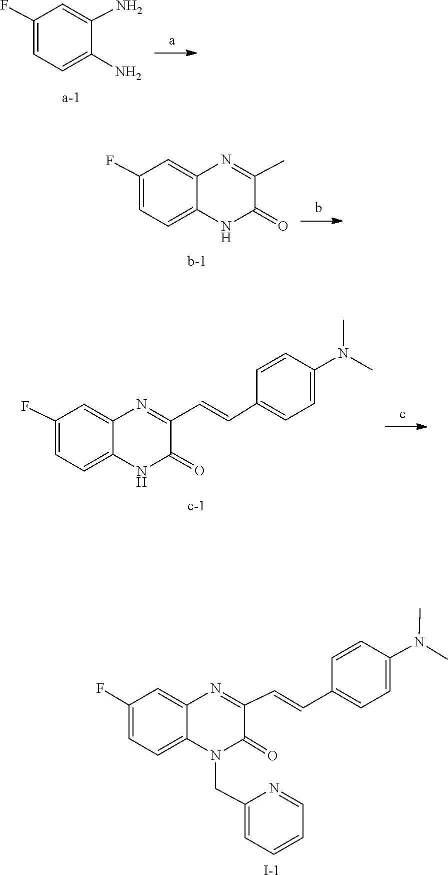

### Step a: Prepare Intermediate b-1

1.26 g (10 mmol) of 4-fluoro-o-phenylenediamine (a-1) was dissolved in 10 ml of anhydrous ethanol, and 2.32 g (20 mmol) of ethyl pyruvate was added and stirred for 4 h at room temperature. The solvent was removed by vacuum and the solid was recrystallized with methanol to give the product a white solid with a yield of 86%. ESI-MS (positive): 179.0 (M+1)⁺.

### Step b: Prepare Intermediate c-1

1.78 g (10 mmol) intermediates b-1 and 1.49 g (10 mmol) 4-dimethylaminobenzaldehyde were dissolved in 5 ml of acetic acid and catalytic concentration of sulfuric acid, and refluxed for 8 h at nitrogen atmosphere. Following the reaction liquid was poured into ice water and extracted with EA 3 times, the organic phase was washed with brine and dried over anhydrous magnesium sulfate, and finally purified by silica gel column chromatography (petroleum ether:ethyl acetate=6:1) to give the product a red solid with a yield of 65%, ¹H NMR (400 MHz, DMSO-d₆) δ 12.20 (s, 1H), 7.80 (d, J=15.7 Hz, 1H), 7.60 (d, J=7.8 Hz, 1H), 7.47-7.35 (m, 4H), 7.18-7.03 (m, 3H), 2.98 (s, 6H). ESI-MS (positive): 310.1 (M+1)⁺.

### Step c: Prepare Compound I-1

0.31 g (1 mmol) compound c-1 was dissolved in 3 mL of DMF, followed by 0.28 g (2 mmol) potassium carbonate and 0.71 g (2 mmol) 2-bromomethyl pyridine and stirred at room temperature for 8 h. Then the reaction liquid was poured into water and extracted with EA 3 times, the organic phase was washed with brine, dried over anhydrous magnesium sulfate, and purified by silica gel column chromatography (petroleum ether:ethyl acetate=4:1) to give the product as brick red solid in a yield of 18%, ¹H NMR (600 MHz, CDCl₃-d) δ 8.59 (d, J=5.3 Hz, 1H), 8.08 (d, J=16.0 Hz, 1H), 7.79 (dd, J=8.9, 5.9 Hz, 1H), 7.65-7.54 (m, 4H), 7.25 (s, 1H), 7.23-7.14 (m, 2H), 7.04-6.96 (m, 1H), 6.71 (d, J=8.7 Hz, 2H), 5.58 (s, 2H), 3.03 (s, 6H). ¹³C NMR (150 MHz, CDCl₃-d) δ 161.83 (d, J=249.5 Hz), 154.54, 151.39 (d, J=3.4 Hz), 150.59, 148.92, 138.12, 136.54, 132.75 (d, J=11.6 Hz), 130.39 (d, J=10.2 Hz), 130.08, 128.97, 123.91, 122.26, 121.42, 116.16, 111.37, 111.22, 111.07, 101.18, 47.77, 39.60. ESI-MS (positive): 401.2 (M+1)⁺.

Example 2: Preparation of compound I-2 whose structure is shown below:

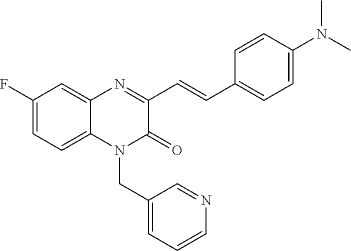 [SMILES: CN(C)c1ccc(/C=C/c2nc3cc(F)ccc3n(Cc3cccnc3)c2=O)cc1]

The preparation method is the same as that of compound I-1, except that 2-bromo-methylpyridine is replaced by 3-bromo-methylpyridine in step c, to obtain a brick red solid in a yield of 20%, ¹H NMR (600 MHz, CDCl₃-d) δ 8.66 (s, 1H), 8.56 (d, J=4.8 Hz, 1H), 8.08 (d, J=16.0 Hz, 1H), 7.82 (dd, J=8.9, 5.9 Hz, 1H), 7.62-7.49 (m, 4H), 7.25 (s, 1H), 7.08-6.97 (m, 1H), 6.88 (dd, J=10.0, 2.5 Hz, 1H), 6.71 (d, J=8.4 Hz, 1H), 5.48 (s, 2H), 3.04 (s, 6H). ¹³C NMR (150 MHz, CDCl₃-d) δ 161.79 (d, J=250.0 Hz), 154.51, 151.40, 150.66, 148.79, 148.10, 138.44, 134.19, 132.17 (d, J=11.2 Hz), 130.84 (d, J=10.0 Hz), 130.17, 130.12, 129.05, 123.78, 123.25, 115.83, 111.37, 111.22, 100.27, 100.09, 43.28, 39.59. ESI-MS (positive): 401.2 (M+1)⁺.

Example 3: Preparation of compound I-3 whose structure is shown below:

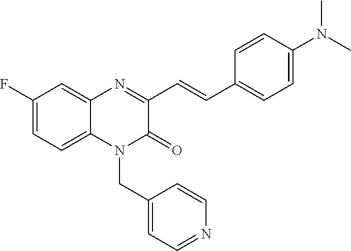 [SMILES: CN(C)c1ccc(/C=C/c2nc3cc(F)ccc3n(Cc3ccncc3)c2=O)cc1]

The preparation method is the same as that of compound I-1, except that 2-bromo-methylpyridine is replaced by 4-bromo-methylpyridine in step c, to obtain a brick red solid in a yield of 20%, 1H NMR (600 MHz, CDCl₃-d) δ 8.58 (d, J=6.2 Hz, 2H), 7.83 (dd, J=8.9, 5.9 Hz, 1H), 7.59 (d, J=8.7 Hz, 2H), 7.55 (d, J=16.0 Hz, 1H), 7.15 (d, J=5.9 Hz, 2H), 7.05-7.01 (m, 1H), 6.75-6.69 (m, 3H), 5.46 (s, 2H), 3.04 (s, 6H). ¹³C NMR (150 MHz, CDCl₃-d) δ 161.77 (d, J=250.3 Hz), 154.40, 151.38, 150.69, 149.85, 143.31, 138.57, 132.13 (d, J=11.1 Hz), 130.84 (d, J=10.0 Hz), 130.08, 129.07, 123.74, 120.97, 115.79, 111.46, 111.36, 111.31, 100.31, 100.13, 44.66, 39.58. ESI-MS (positive): 401.2 (M+1)⁺.

Example 4: Preparation of compound I-4 shown below:

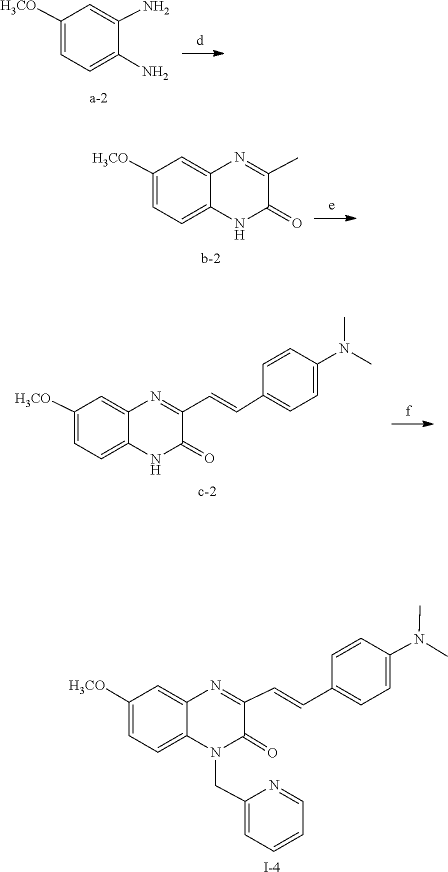

### Step d: Prepare Intermediate b-2

It is prepared by the same method as the compound b-1, except that 4-fluoro-o-phenylenediamine (a-1) is replaced by 4-methoxy-o-phenylenediamine (a-2). The gray solid was obtained with an 81% yield. ESI-MS (positive): 191.0 (M+1)⁺.

### Step e: Prepare Intermediate c-2

The preparation method is the same as that of the compound c-1, except the intermediate b-1 is replaced by the intermediate b-2, and the product is obtained as a red solid with a yield of 55%. 1H NMR (400 MHz, DMSO-d₆) δ 12.29 (s, 1H), 7.86 (d, J=16.1 Hz, 1H), 7.62 (d, J=8.9 Hz, 1H), 7.51 (d, J=8.1 Hz, 2H), 7.31 (d, J=16.3 Hz, 1H), 6.89 (d, J=8.9 Hz, 1H), 6.73 (s, 3H), 3.81 (s, 3H), 2.96 (s, 6H). ESI-MS (positive): 322.1 (M+1)⁺.

### Step f: Prepare Compound I-4

The preparation method is the same as that of the compound I-1, except that the intermediate c-1 is replaced by the reactant c-2, and the brick red solid is obtained with a yield of 17%. ESI-MS (positive): 413.2 (M+1)⁺.

Example 5: Preparation of compound I-6, as shown below:

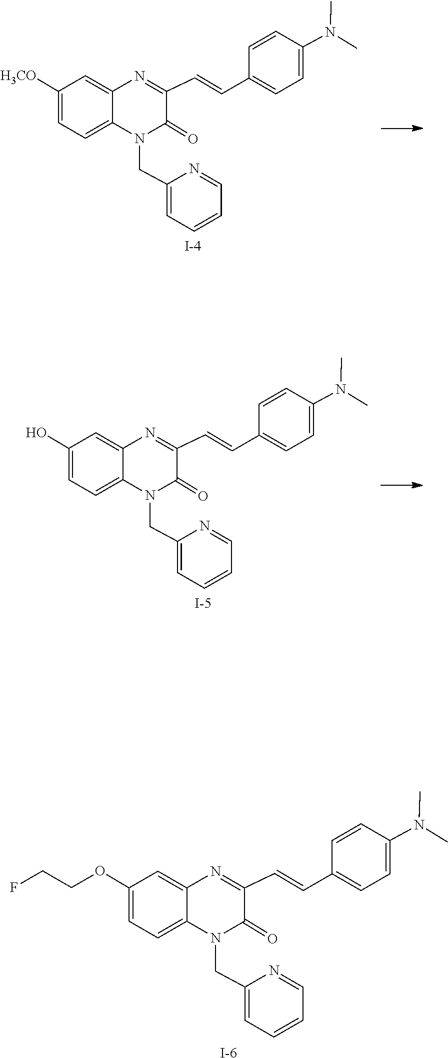

0.40 g compound I-4 was dissolved in 2 mL boron tribromide solution and then stirred at room temperature for 3 hours. After that, the solvent was evaporated to obtain I-5. Then 3 mL N, N-dimethylformamide, 0.08 g potassium carbonate, and 0.25 g 1-bromo-2-fluoroethane were added and the mixture was stirred at room temperature for 8 h. Following 20 ml of water was added, and extracted with EA. The organic phase was washed with brine, dried over anhydrous magnesium sulfate, and purified by silica gel column chromatography (ethyl acetate:petroleum ether=1:1) to obtain a light yellow solid with a yield of 9%, 1H NMR (600 MHz, CDCl₃-d) δ 8.59 (d, J=4.8 Hz, 2H), 8.00 (d, J=16.4 Hz, 1H), 7.83 (d, J=9.2 Hz, 1H), 7.70-7.49 (m, 3H), 7.12 (d, J=5.5 Hz, 2H), 6.97 (d, 4.4 Hz, 1H), 6.71 (d, J=8.4 Hz, 2H), 6.54 (d, J=2.4 Hz, 1H), 5.50 (s, 2H), 4.70 (d, J=22.9 Hz, 2H), 4.28 (d, J=16.7 Hz, 2H), 3.03 (s, 6H). ESI-MS (positive): 445.2 (M+1)⁺.

Example 6: Preparation of compound I-9, as shown below:

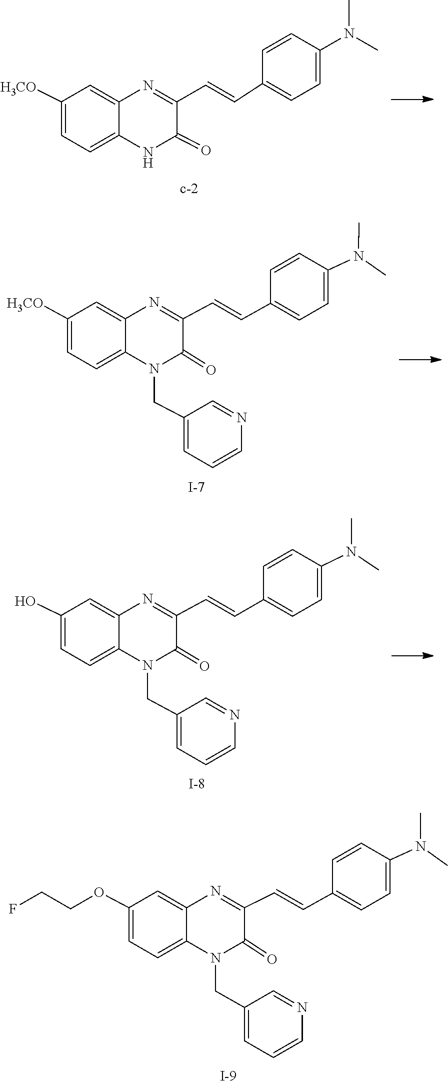

I-7 is prepared in the same way as that of the compound I-4, except that 2-bromomethyl pyridine is replaced by 3-bromomethyl pyridine. The preparation of the following steps is the same as that of I-6. Brick red solid was obtained with a yield of 12%, 1H NMR (600 MHz, CDCl₃-d) δ 8.55 (d, J=5.1 Hz, 2H), 8.02 (d, J=14.8 Hz, 1H), 7.73-7.52 (m, 4H), 7.02 (d, −6.95 (m, 3H), 6.75-6.66 (m, 3H), 5.50 (s, 2H), 4.70 (d, J=37.2 Hz, 2H), 4.11 (d, J=30.5 Hz, 2H), 3.00 (s, 6H). ESI-MS (positive): 445.2 (M+1)⁺.

Example 7: Preparation of compound I-12, as shown below:

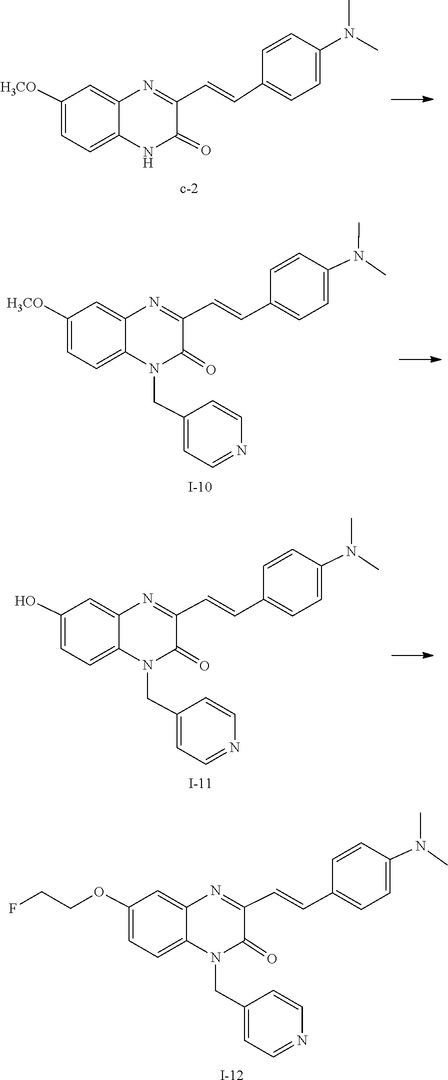

I-10 is prepared in the same way as that of the compound I-4, except that 2-bromomethyl pyridine is replaced by 4-bromomethyl pyridine. The preparation of the following steps is the same as that of I-6. Brick red solid was obtained with a yield of 14%, 1H NMR (600 MHz, CDCl₃-d) β 8.57 (d, J=5.2 Hz, 2H), 8.04 (d, J=16.0 Hz, 1H), 7.79 (d, J=8.8 Hz, 1H), 7.69-7.51 (m, 3H), 7.15 (d, J=5.1 Hz, 2H), 6.91 (dd, J=8.9, 2.4 Hz, 1H), 6.71 (d, J=8.4 Hz, 2H), 6.54 (d, J=2.4 Hz, 1H), 5.48 (s, 2H), 4.72 (d, J=47.4 Hz, 2H), 4.18 (d, J=28.1 Hz, 2H), 3.03 (s, 6H). ¹³C NMR (150 MHz, CDCl₃-d) δ 158.39, 154.70, 150.49, 149.72, 143.83, 137.42, 132.30, 130.44, 128.83, 128.52, 124.03, 121.04, 116.37, 111.40, 110.28, 99.22, 80.98 (d, J=171.7 Hz), 66.92 (d, J=20.4 Hz), 44.47, 39.60. ESI-MS (positive): 445.2 (M+1)⁺.

Example 8: Preparation of compound I-15, as shown below:

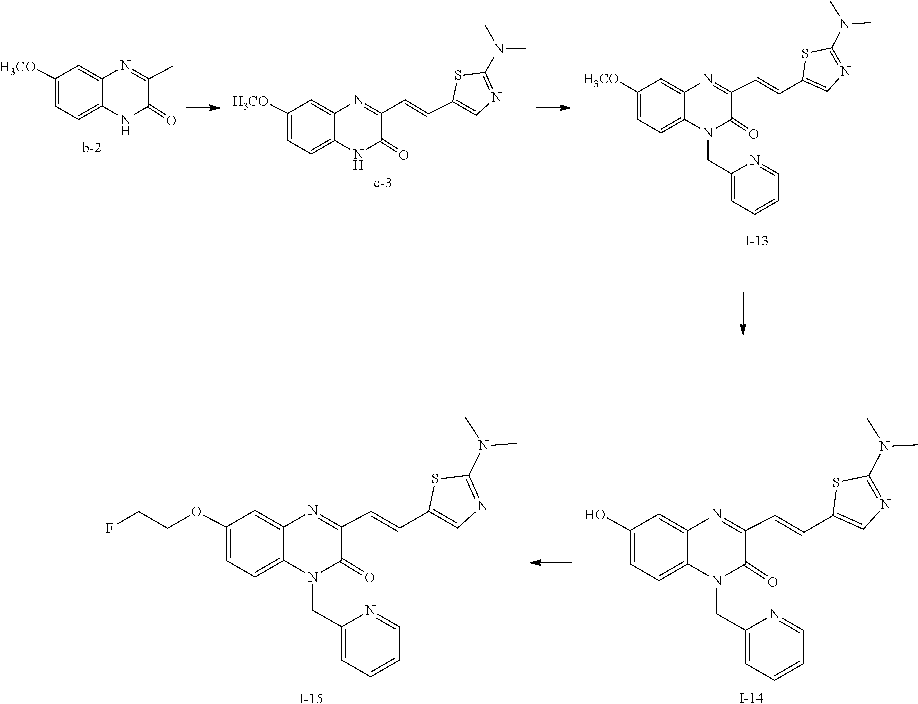

The preparation method of I-13 is the same as that of I-4, except that 4-dimethylaminobenzaldehyde is replaced by 2-dimethylaminothiazole-5-formaldehyde in step e, and the subsequent steps are the same as the preparation of I-6. Red solid was obtained with a yield of 21%, ¹H NMR (600 MHz, DMSO-d₆) δ 8.02 (dd, 1H), 7.57 (s, 1H), 7.81-7.67 (m, 2H), 7.39-7.27 (m, 2H), 7.01-6.85 (m, 4H), 5.61 (s, 2H), 4.71 (m 2H), 4.26 (m, 2H), 3.13 (s, 6H). ¹³C NMR (150 MHz, DMSO-d₆) 170.38, 158.67, 154.97, 154.23, 149.02, 145.42, 136.96, 133.32, 130.04, 127.99, 127.49, 125.45, 122.50, 121.73, 117.35, 111.32, 99.83, 81.69 (d, J=166.9 Hz), 67.49, 55.53, 46.61. ESI-MS (positive): 451.2 (M+1)⁺.

### [Labeling of Radionuclides]

Various radionuclides can be labeled by conventionally known methods. The preparation of (¹⁸F) I-1, (¹⁸F) I-15, and (¹¹C) I-13 is used as examples to illustrate the method of labeling ¹⁸F and ¹¹C, respectively. Other radioactive tracers can be prepared in the same way.

Example 9: Synthesis of a radioactive tracer (¹⁸F)I-1

As shown in the scheme below, a variety of precursor compounds can be labeled with radionuclide ¹⁸F. The synthesis of three precursors (containing nitro, bromine, or borate) are given below as examples but not limited to.

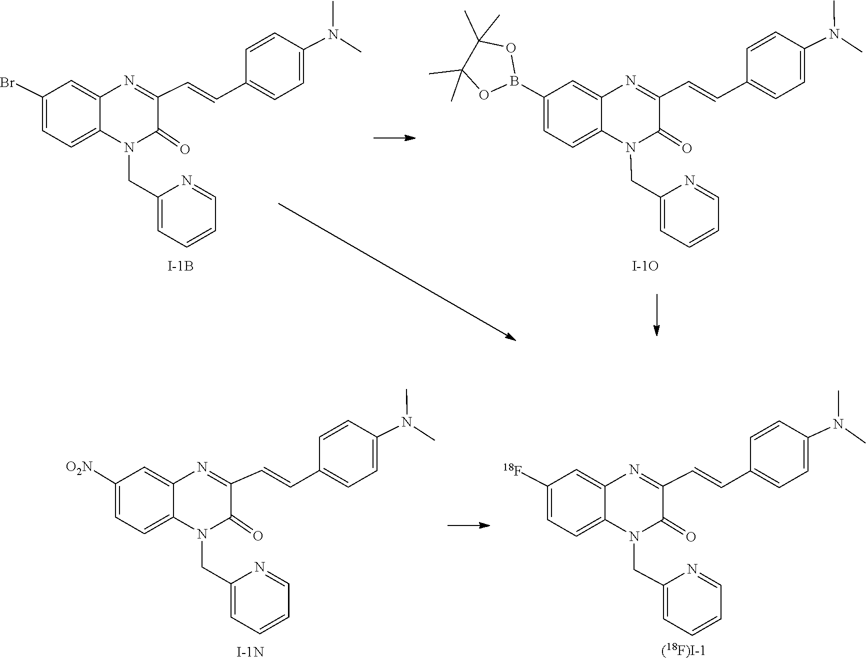

The nitro-containing precursor compound I-1N and the bromo-containing precursor compound I-1B were prepared the same as that described in Example 1, except replacing 4-fluoro-o-phenylenediamine with 4-nitro-o-phenylenediamine and 4-bromo-o-phenylenediamine, respectively. Further, the brominated precursor I-1B was coupled with pinacol borate catalyzed by palladium to prepare the more active borate-containing precursor compound I-10. All three precursors react with radioactive K¹⁸F to form a radioactive tracer (¹⁸F) I-1.

Method 1: Synthesis from the precursor compound I-10 containing borate. ¹⁸F is produced by a cyclotron and eluted into the reaction tube by K₂₂₂/K₂CO₃ elution from bottle 1 after QMA adsorption, and evaporated in a nitrogen atmosphere at 116° C. The solution in bottle 2 (2 mL acetonitrile) was injected into the reaction tube, and the water was removed by azeotrope evaporation in a nitrogen atmosphere at 116° C. After cooling of reaction tube for 60 s, the solution in bottle 3 (8 mg precursor compound I-10 dissolved in 1 mL of DMF) was injected into the reaction tube at 115° C. and kept for 30 min. After cooling for 100 s (≤40° C.), the solution in bottle 4 (10 mL of distilled water) was injected into the reaction tube for dilution, then transferred to the C-18 column followed by eluted with 2.5 mL anhydrous ethanol, which was diluted with normal saline to less than 10% ethanol. The (¹⁸F)I-1 solution for injection was finally obtained by filtration with a 0.22 μm filter membrane. The success of the radio-labeling was proved by comparing the consistency of the retention time between the prepared radio-labeled compound and the non-radioactive compound I-1 by HPLC.

Method 2: Synthesis from the nitro-containing precursor compound I-1N. The (¹⁸F) fluoride ions were dissolved into a 50% acetonitrile solution (0.4 mL) containing K₂₂₂ (Kryptofix 222) (7.5 mg) and potassium carbonate (2.77 mg). The solution was introduced into the reaction vessel and heated under nitrogen to dry. Then anhydrous acetonitrile (0.1 mL) was added for azeotropic distillation to dry the reaction vessel fully. A DMSO (300 μL) solution containing the nitro-containing precursor compound I-1N (1 mg) was added to the reaction vessel and heated at 110° C. for 10 minutes. After cooling, (¹⁸F)I-1 was purified by HPLC.

Similarly, the brominated precursor compound I-1B can also be labeled with ¹⁸F to synthesize (¹⁸F) I-1 under similar conditions in method 2.

Example 10: Synthesis of radioactive tracer probe (¹⁸F)I-15

As shown in the scheme below, the radioactive tracer (¹⁸F) I-15 can be prepared by direct oxy alkylation of its precursor compound I-14 with the bromo-alkane ¹⁸F—CH₂CH₂—Br which has been labeled by ¹⁸F. Or I-14 reacted with 1, 2-bis(toluene sulfonyloxy) ethane to produce precursor compound I-15T containing leaving group TsO—; I-14 can also be reacted with ethylene oxide to form compound I-150 containing a terminal hydroxyl group, following reacted with p-methylbenzene sulfonyl chloride (TsCl) or methane sulfonyl chloride (MsCl) under alkaline conditions to form a precursor compound (such as I-15T) with label site containing a leaving group (such as TsO- or MsO—). Finally, I-15T reacted with radioactive K¹⁸F to form a radioactive tracer (¹⁸F)I-15.

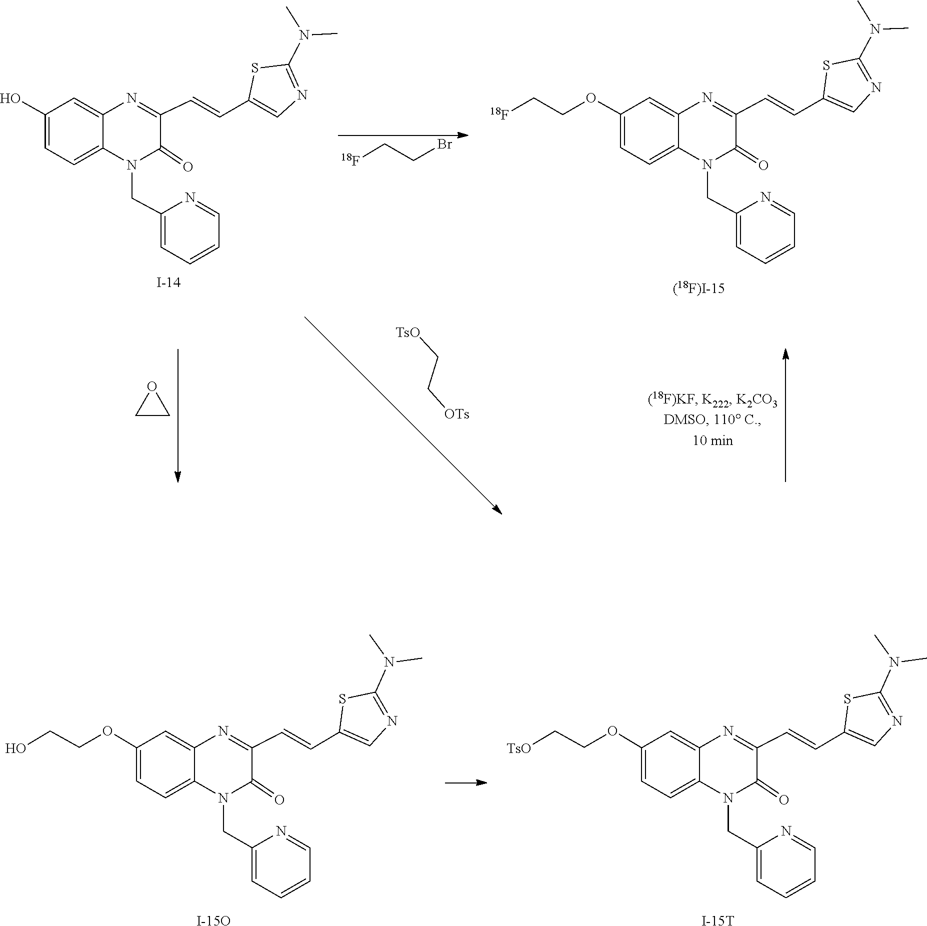

Example of preparation of the precursor compound I-15T: 0.16 g compound I-14 dissolved in 5 mL N, N-dimethylformamide with adding 0.11 g potassium carbonate (2 eq) and 0.3 g compound 1, 2-bis(toluene sulfonoxyl) ethane (2 eq), and the mixture stirred overnight at room temperature. 20 mL water was added to the mixture and extracted with ethyl acetate. The organic phase was dried over anhydrous magnesium sulfate and the crude product was purified by silica gel column chromatography (PE:EA=1:1).

The following is an example of the preparation of (¹⁸F) I-15 from the precursor compound I-15T containing the TsO-group.

The (¹⁸F) fluoride ions were dissolved into a 50% acetonitrile solution (0.4 mL) containing K₂₂₂ (Kryptofix 222) (7.5 mg) and potassium carbonate (2.77 mg). The solution was introduced into the reaction vessel and heated under nitrogen to dry. Then anhydrous acetonitrile (0.1 mL) was added for azeotropic distillation to dry the reaction vessel fully. A solution of DMSO (300 μL) dissolved with precursor I-15T (1.0 mg) was added to the reaction vessel and heated at 110° C. for 10 minutes. After cooling, (¹⁸F)I-15 was isolated and purified by HPLC (C-18 column).

Example 11: Synthesis of a radioactive tracer (¹¹C)I-13 is performed away from light, as shown in the scheme below. (¹¹C) Iodine methane was added to 300 μL of dimethyl sulfoxide (DMSO) solution dissolved with I-14 (2 mg) at room temperature. After the reaction mixture was heated at 120° C. for 5 minutes, the mixture was cooled and purified by HPLC. The component of (¹¹C)I-13 was recovered into a flask containing ethanol (300 μL), 25% ascorbic acid (100 μL), and Tween80 (75 μL). The solvent was removed by vacuum distillation. The residue was dissolved in normal saline (3 mL, pH 7.4) to obtain (¹¹C)I-13 as an injection solution.

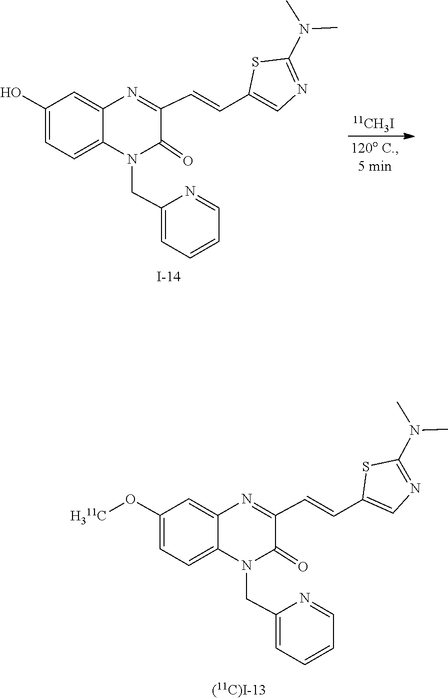

### [Binding Affinity of the Compounds to α-Synuclein Aggregates]

The binding affinity of the compound of the invention to human α-synuclein aggregates is determined by the fluorescence method described below.

### (1) Preparation of α-Synuclein Monomer

1 μL ampicillin anti-plasmid with α-synuclein in the correct sequence was mixed with 100 μL BL21 (DE3) receptive cells, cooled in an ice bath, added in 600 μL LB medium, and cultured in a 220 rpm shaking bed at 37° C. for 90 min. 150 μL cultured bacterial solution was added to the sterilized dish coated with ampicillin medium evenly. Positive clonal colonies were selected and added to the configured ampicillin medium and cultured in an incubator at 37° C. The cultured positive clones were poured into 1 L of 2×YT medium and cultured in a 220 rpm shaker at 37° C. to make OD 600 0.6 and then cooled to 18° C. Each medium was induced to culture for 16 hours by adding 1 ml of IPTG (500 mM).

The bacteria were centrifuged at high speed for 30 min after ultrasonic. The supernatant was collected to remove DNA and hetero-proteins by Ni-NTA chromatography. α-synuclein monomer was purified by molecular exclusion chromatography and the purity was verified by SDS-PAGE discontinuous electrophoresis.

### (2) Preparation of α-Synuclein Aggregates

α-synuclein monomers were prepared into a buffer solution containing 1×PBS, where the final protein concentration was 100 μM (about 5 mg/mL), and incubated in a 1000 rpm shaker at 37° C. for 7 days to obtain α-synuclein aggregates. The initial and final concentrations of the monomer protein were determined by the BCA method.

The prepared α-synuclein aggregates, also known as preformed fibrils (PFFs), are used for protein affinity testing, construction of cell model, and testing of the invention.

### (3) Binding Activity Test of the Compound

10 mM mother solution of DMSO was prepared from 1 mg of the compound, then diluted to 20 μM with PBS, and made gradient dilution 7 times (3× each dilution); 30 μL of test compound was added to the 384-well plate, following the experimental group was added to 30 μL α-synuclein aggregates (3 μM), and the control group was added to the same amount of PBS. After the 384-well plate was incubated shaking at 50 rpm at room temperature for 1 hour, the plate was taken out to detect the maximum absorption and emission wavelengths of the compound with an ELISA Microplate Reader, and the fluorescence values were detected at these wavelengths, too. The fluorescence changes in different concentrations of compounds were calculated using the experimental group data minus the control group data, and the protein-binding affinity of compounds was obtained using the Saturation binding module of GraphPad Prism.

The binding affinity of the Formula I compound to α-synuclein aggregates is determined by the above method, and the dissociation equilibrium constant K_{d} is shown in Table 1.

TABLE 1
Binding affinity (K_{d}) of partial Formula I compound
of the invention on human α-synuclein aggregates

| Example compound | K_{d} (μM) |
|---|---|
| I-1 | *** |
| I-2 | * |
| I-3 | * |
| I-6 | ** |
| I-9 | * |
| I-12 | ** |
| I-15 | *** |

* 1.0~0.5 μM;
** 0.5~0.2 μM;
*** 0.2~0.1 μM.

### [Immunofluorescence Imaging of Cell Models]

SH-SY5Y cells belong to the SK—N—SH cell line and are a kind of human neuroblastoma cells. These cells can express a variety of important proteins of neurons, such as dopaminergic transporters, dopamine hydroxylase, and tyrosine hydroxylase, so they are often used in the study of the mechanism and pharmacodynamic evaluation of Parkinson's disease. The prepared α-synuclein aggregates (PFFs) are co-incubated with SH-SY5Y cells and endocytosed into cells after 12 hours through endocytosis. The cells were further incubated with α-synuclein antibodies and compounds successively, and observed by microscope after washing with PBS. The detailed operations are as follows.

SH-SY5Y cells were cultured in a high-glucose DMEM medium (containing 10% Gibco fetal bovine serum). After 5 times of resuscitation and passage, the cell state tended to be stable. Then PFFs was added to the medium and fluorescent staining was performed after 48 h. After sucked the cell culture solution and cleaned with PBS three times, 0.3% Triton X-100 was added and incubated for 10 min. After washing with PBS, 10% goat serum was added and closed for 1 h; After cleaning with PBS, primary antibody (1:1000, 610786, BD Biosciences) was added and incubated at 4° C. overnight. After cleaning with PBS, secondary antibodies (1:1000, goat-anti-rabbit Alex Fluor 594, and goat-anti-mouse Alex Fluor 488, Invitrogen) were added and incubated at room temperature for 2 h. Finally, the compound of the invention is added and incubated at room temperature for 1 h. After cleaning with PBS, the tablet is sealed and photographed by a laser confocal microscope.

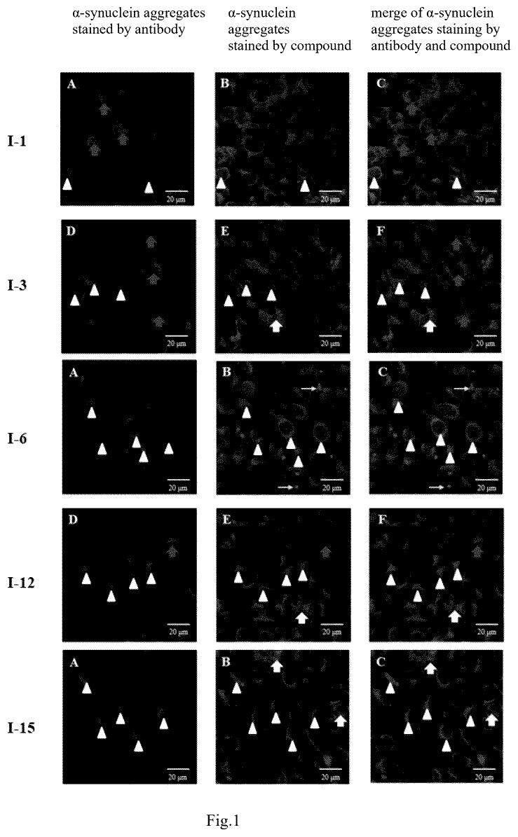

The results are shown in FIG. 1, indicating that all the Formula I compounds tested can well bind to α-synuclein aggregates in SH-SY5Y cells, especially compounds I-6, I-12, and I-15 show excellent binding affinity and specificity without almost non-specific binding.

### [Optical Imaging in Patient's Brain]

Staining and Imaging of Brain Slices from Patients of Dementia with Lewy Body (DLB)

Brain slices from patients of dementia with Lewy bodies were taken from the amygdala of a deceased 75-year-old man in stage 2 of dementia with Lewy bodies. Frozen sections of the amygdala with rich α-synuclein were performed with a thickness of 20 μm.

Compound I-15 was diluted to 30 μM with PBS solution containing 50% EtOH, following incubated with the fresh frozen brain sections at room temperature for 30 minutes, then washed with 50% ethanol solution for 5 minutes and ultra-pure water twice for 3 minutes each time in sequence. After the sections were buried with an embedding agent (VECTASHIELD H-1000, Vector Laboratories), images of the lesion accumulation area on the sections were obtained by fluorescence microscopy. Analysis software (Image J) was used to quantify the fluorescence radiance of both the lesion area and the area of the non-forming lesion (background) to evaluate binding selectivity.

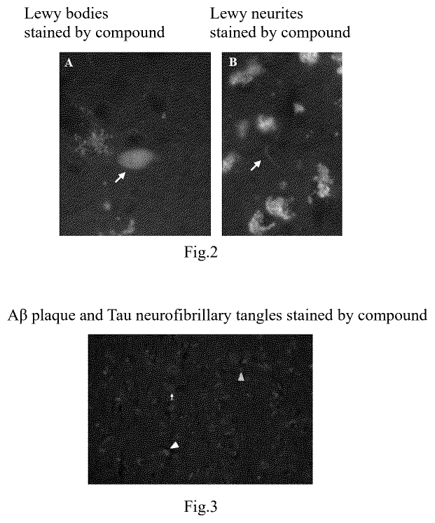

Fluorescent image results in FIG. 2 show that compound I-15 can stain Lewy bodies and Lewy neurites in brain slices of patients with Lewy body dementia, indicating strong binding affinity to α-synuclein lesions in the brain of patients.

Staining and Imaging of Brain Slices from Patients with Alzheimer's Disease (AD)

Brain slices of the superior temporal gyrus were taken from a stage 3 Alzheimer's patient after the death. The dewaxed brain tissue was fixed in a 10% neutral buffer of formalin solution, embedded with paraffin, and then sliced with a thickness of 6 μm. The staining method is the same as the above method for DLB patients. As shown in FIG. 3, the fluorescence image results showed that compound I-15 could also detect the original Aβ plaques, Aβ dense core plaques, and Tau neurofibrillary tangles in the brain slices of AD patients, but did not bind to Tau neurofibrillary filaments. The staining signal of the compound was much weaker in AD brain slices than in DLB brain slices, indicating that the binding affinity of I-15 to both Aβ and Tau histology was very weak.

According to the results of FIG. 2 and FIG. 3, the binding of compound I-15 to α-synuclein pathological tissue was significantly stronger than that to Aβ and Tau pathological tissue, indicating that it had a very good binding selectivity for α-synuclein aggregates.

### [Blood-Brain Barrier Permeability Test]

The compound of the invention is injected into a rat tail vein to determine the blood-brain barrier permeability in vivo according to the following method.

Dissolve the tested compound in DMSO, and add castor oil and PBS for dilution (DMSO:castor oil:PBS=1:1:8). SD rats were weighed and given 5 mg/kg in the tail vein. 500 μL of blood was taken 20 min after administration after anesthetized with isoflurane. Cardiac perfusion was performed with 200 mL PBS and stopped until the organ faded, following brain tissue was got out and washed with PBS.

After centrifuging the extracted blood at 9000 rpm for 5 min, 200 μL supernatant was taken, and added 800 μL methanol. After centrifuging at 14000 rpm for 10 min, the supernatant was taken and filtered through a 0.22 μm filter membrane and stored at −80° C. for use. 2 mL PBS and 2 mL methanol were added to about 0.5 g of brain tissue to make a tissue homogenization, and then 1 mL homogenate was taken out and centrifuged at 14,000 rpm for 10 min after 2 mL methanol was added, 1 mL supernatant was taken through 0.22 μm filter membrane and stored at −80° C. for use. The concentration of compounds in the above supernatant of blood and brain homogenate were checked by LC-MS/MS, respectively.

A brain/blood ratio of <0.1, 0.1-0.3, or >0.3 indicates blood-brain barrier penetration of weak, moderate, or good, respectively. The test results show that the brain/blood ratio of the compounds I-6, I-12, and I-15 of the invention is close to or greater than 1.0, proving that they all have good blood-brain barrier permeability. Since all the compounds of the invention have similar structures and their clogP values are mostly between 1.0 and 3.0, it can be predicted that other compounds of the invention should also have acceptable blood-brain barrier permeability.

### INDUSTRIAL PRACTICALITY

Because of good binding/staining affinity to α-synuclein aggregates, the compounds of the present invention and its composition are extremely important for the early detection, treatment, and prevention of severe diseases such as Parkinson's disease, which is one of the most important medical difficulties at present. The compound of the invention can be used as an imaging tracer to visualize the accumulation of α-synuclein, thus providing early diagnosis and disease progression information for various neurodegenerative diseases, such as Parkinson's disease (PD), Parkinson's disease dementia (PDD), dementia with Lewy body (DLB), multiple system atrophy (MSA), etc.

1. A compound represented by general Formula I, its pharmaceutically acceptable salt or solvate, which can be used as a tracer for the imaging diagnosis of α-synuclein accumulation diseases, 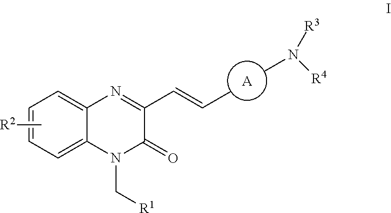
wherein,
R¹ is 5~6 membered aryl;
R² is selected from halogen, nitro, hydroxyl, C₁₋₄ alkoxyl, and halogenated C₁₋₄ alkoxyl,
wherein the halogen is selected from fluorine, chlorine, bromine, or iodine;
R³ and R⁴ are independently selected from hydrogen, and C₁₋₃ alkyl;
Ring A is selected from phenyl ring, 5- and 6-membered aromatic heterocyclic ring.

2. A compound, its pharmaceutically acceptable salt or solvate thereof according to claim 1, wherein R¹ is preferably pyridyl.

3. A compound, its pharmaceutically acceptable salt or solvate thereof according to claim 1, wherein R³ and R⁴ are preferably methyl.

4. A compound, its pharmaceutically acceptable salt or solvate thereof according to claim 1, wherein ring A is preferably phenyl ring or thiazole ring.

5. A compound, its pharmaceutically acceptable salt or solvate thereof according to claim 1, wherein one or more atoms of the compound of Formula I are the radioisotopes of that atom, of which preferably taken from ¹¹C, ¹³N, ¹⁵O, ¹⁸F, ⁷⁶Br, ¹²³I, ¹²⁵I, and ¹³¹I.

6. The compound according to claim 5, a pharmaceutically acceptable salt or a solvate thereof, wherein the compound represented by Formula I is selected from the following structures: 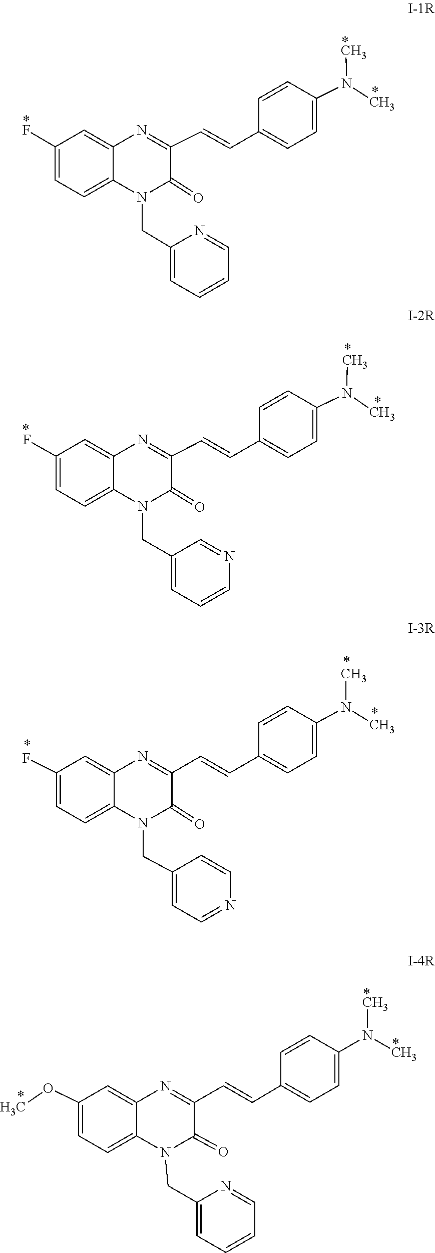 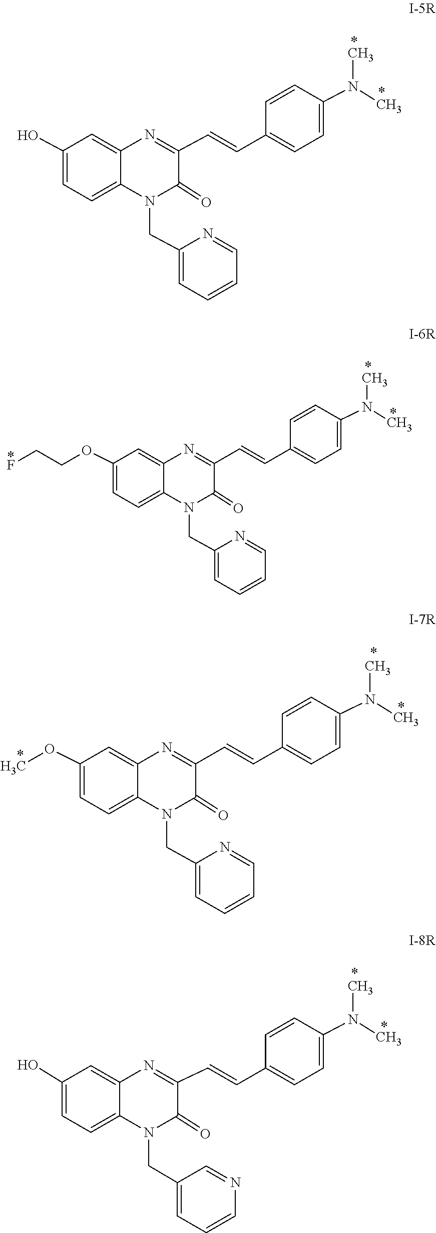 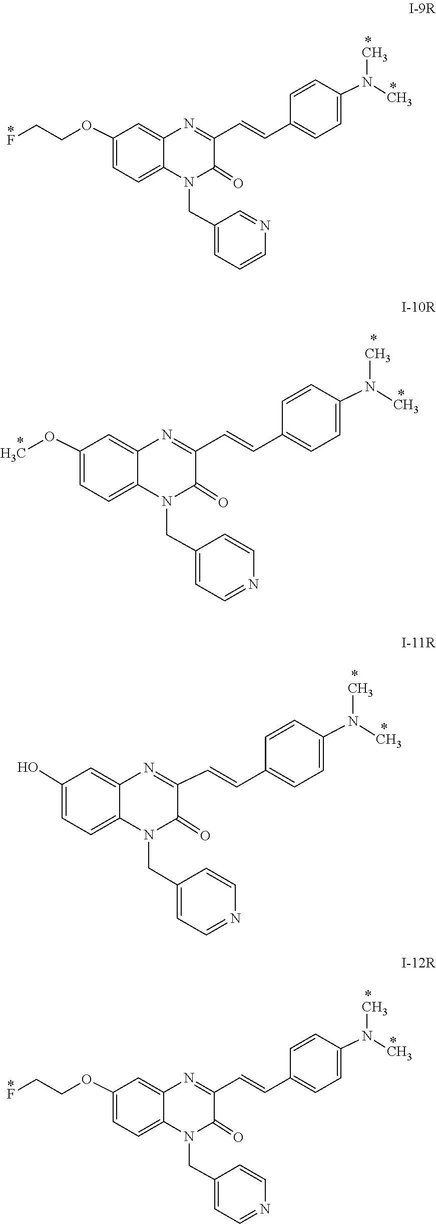 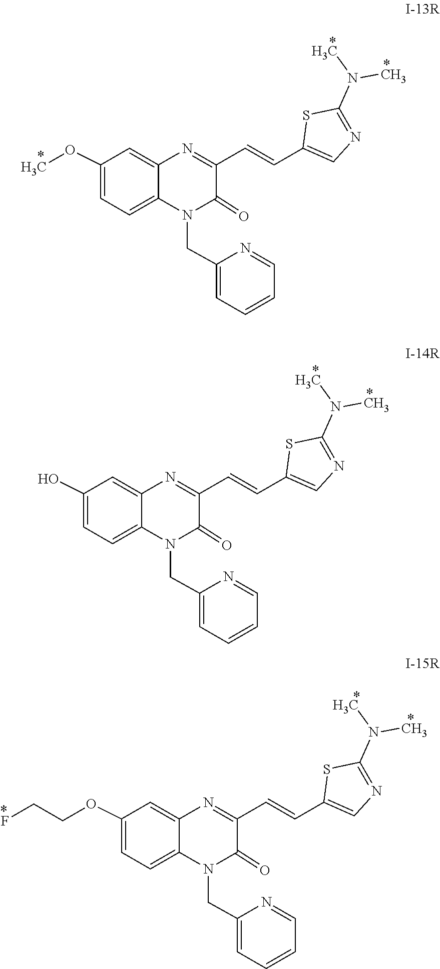
wherein one of the atoms marked with * is a radioisotope of that atom at least.

7. A compound selected from the following structures, which is used as a precursor for the synthesis of the compound according to claim 6, 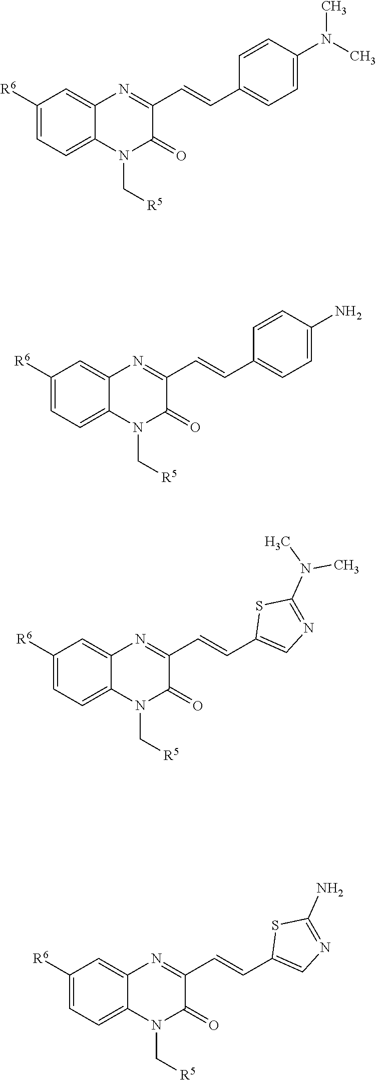
wherein, R⁵ is pyridyl; R⁶ is independently selected from hydroxyl, fluorine, bromine, iodine, nitro, borate group, TsO—(CH₂)m-O—, MsO—(CH₂)m-O—, where m is an integer from 2 to 4.

8. (canceled)

9. (canceled)

10. The use of a compound represented by general Formula I, its pharmaceutically acceptable salt, or solvate thereof according to claim 1 in the preparation of a tracer for imaging diagnosing α-synuclein accumulation diseases, wherein the compound or its pharmaceutically acceptable salt or solvate thereof can bind to α-synuclein aggregates.
